(19) United States
(12) Patent Application Publication
(10) Pub. No.: US 2012/0302546 A1
(43) Pub. Date: Nov. 29, 2012

(54) NOVEL SULFAMIDES EXHIBITING NEUROPROTECTIVE ACTION AND METHODS FOR USE THEREOF

(75) Inventors: Garry Robert Smith, Royersford, PA (US); Douglas Eric Brenneman, North Wales, PA (US); Allen B. Reitz, Lansdale, PA (US); Yanming Du, Cheshire, CT (US)

(73) Assignees: FOX CHASE CHEMICAL DIVERSITY CENTER, INC., Doylestown, PA (US); ADVANCED NEURAL DYNAMICS, INC., Doylestown, PA (US)

(21) Appl. No.: 13/576,556

(22) PCT Filed: Feb. 3, 2011
(86) PCT No.: PCT/US11/23536

§ 371 Date: Aug. 1, 2012

Related U.S. Application Data
(60) Provisional application No. 61/301,540, filed on Feb. 4, 2010.

(51) Int. Cl. C07C 307/06; A61P 25/28; A61K 31/397; C07D 333/58; A61K 31/381; C07D 409/04; A61K 31/16; A61P 25/08

(52) U.S. Cl. 514/210.19; 564/79; 514/600; 549/58; 514/443; 548/952

(57) ABSTRACT

Pharmaceutical compositions of the invention include sulfamide derivatives having a disease-modifying action in the treatment of diseases associated with excitotoxicity and accompanying oxidative stress that include epilepsy, Alzheimer's disease, and any neurodegenerative disease involving glutamate toxicity.

### CROSS-REFERENCE TO RELATED APPLICATIONS

This application claims the benefit of U.S. Provisional Application No. 61/301,540 filed Feb. 4, 2010, which is herein incorporated by reference in its entirety.

### STATEMENT OF FEDERALLY FUNDED RESEARCH

The U.S. Government has a paid-up license in this invention and the right in limited circumstances to require the patent owner to license others on reasonable terms as provided for by the terms of grant number 5R43NS066537-02 awarded by the National Institute of Neurological Disorders And Stroke.

### FIELD OF THE INVENTION

The present disclosure relates to compositions and methods for neuroprotection, useful in the treatment of epilepsy and related conditions. The present disclosure is more particularly related to novel chemotype, neuroprotective compounds that have been discovered and identified for the treatment of neurodegenerative disease, epilepsy, and other diseases that involve excessive glutamate in their etiology.

### BACKGROUND OF THE INVENTION

The statements in this section merely provide background information related to the present disclosure of the invention and do not constitute prior art. There currently are approximately 30 marketed antiepileptic drugs and at least another 20 compounds in Phase 1-3 drug development. All possess unwanted CNS side effects. In addition to the obvious need for drugs with lower toxicity and higher therapeutic index, an emerging concept is that neuroprotection may be a strategy for both limiting neural damage associated with seizures and providing long-term antiepileptogenesis, the ultimate goal for this therapeutic area.

There are presently no validated molecular targets which allow for effective screening in vitro for anti-seizure compounds. Although no definitive molecular target/mechanism of action exists for antiepileptic drugs, many modulate various ion channels (Na⁺, K⁺ and Ca²⁺) and amino acid receptors (NMDA, AMPA/KA and GABA. Therefore, the strategy of the present inventors has been to make novel analogues of existing anticonvulsants and to optimize these novel compounds for neuroprotection.

For the purposes of the present invention with respect to neuroprotection, activity in hippocampal cultures treated with glutamate is the experimental model which has been employed as the best predictor of efficacy, rather than a singular molecular target. With a focus on a novel chemotype, neuroprotective compounds have been identified by the present inventors for the treatment of neurodegenerative disease, epilepsy, and other diseases that involve excessive glutamate in their etiology.

In accordance with the present invention the neuroprotective action of novel hydroxyethyl sulfamides has been discovered, that prevents neuronal cell death and damage from glutamate toxicity. Such neuroprotective action predicts a potential disease-modifying action in epilepsy, Alzheimer's disease and other therapeutics indications relevant to glutamate toxicity.

### SUMMARY OF THE INVENTION

According to the present invention there are now provided novel hydroxyethyl sulfamides exhibiting neuroprotective action that mitigate or prevent neuronal cell death and damage from glutamate toxicity. The experiments leading to the present invention predict a potential disease-modifying action of the compounds provided in epilepsy. Alzheimer's disease and other therapeutics indications relevant to glutamate toxicity.

In one embodiment, the present invention is directed toward novel sulfamide derivatives, compounds of formula (I),

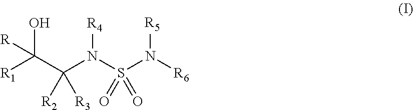

including hydrates, solvates, pharmaceutically acceptable salts, prodrugs and complexes thereof,wherein R, R₁, R₂, R₃, R₄, R₅, and R₆ are as defined herein.

Illustrative of a preferred embodiment of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

There is further provided by the invention a method for treating a subject to prevent neuronal cell death and damage from glutamate toxicity comprising administering a physiologically effective amount of a pharmaceutical composition comprising a pharmaceutically acceptable carrier and any of the compounds described above.

Further areas of applicability of the present invention will become apparent to those skilled in the art from the description provided herein. It should be understood that the description and specific examples are intended for purposes of illustration only and are not intended to limit the scope of the present disclosure.

### BRIEF DESCRIPTION OF THE DRAWING AND FIGURES

FIG. 1 shows the effect of compound designated II in accordance with the present invention on CFDA viability assay on Day 20 Hippocampal Cultures: Neuroprotection from 30 μM Glutamate.

FIG. 2 shows the effect of compound designated III in accordance with the present invention on CFDA viability assay on Day 20 Hippocampal Cultures: Neuroprotection from 30 μM glutamate.

FIG. 3 shows the effect of compound designated IV in accordance with the present invention on Day 20 Hippocampal Cultures: CFDA assay of neuronal viability.

FIG. 4 shows the effect of compound designated II in accordance with the present invention on cell death produced by acute glutamate: assay with propidium iodide on Day 20 Hippocampal Cultures.

FIG. 5 shows the effect of compound designated III in accordance with the present invention on propidium iodide assay of Day 15 Hippocampal Cultures: neuroprotection from 30 μM glutamate.

### DETAILED DESCRIPTION OF THE INVENTION

The present invention provides novel hydroxyethyl sulfamides exhibiting neuroprotective action that are effective to mitigate or prevent neuronal cell death and damage from glutamate toxicity.

The present invention is directed toward novel sulfamide derivatives, compounds of formula (I),

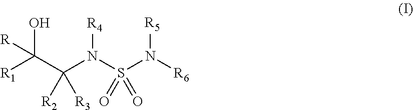

including hydrates, solvates, pharmaceutically acceptable salts, prodrugs and complexes thereof, wherein:
R is selected from the group consisting of optionally substituted aryl, heterocyclyl, heteroaryl, aliphatic, where R may be substituted by 0-5 moieties including, but not limited to aliphatic, hydroxyl, alkoxy amino, nitro, cyano, and halogen.
R₁, R₂, R₃, R₄, R₅ and R₆ are each independently selected from the group consisting of hydrogen and aliphatic;
R₁ and R₂ or R₃ or R₄ may be part of a ring of 4-8 atoms, which may include one or more C, N, O, and/or S.
R₂ and R₃ may be part of a ring of 4-8 atoms, which may include one or more C, N, O, and/or S.
R₂ or R₃ and R₄ may be part of a ring of 4-8 atoms, which may include one or more C, N, O, and/or S.
R₄ and R₅ or R₆ may be part of a ring of 4-8 atoms, which may include one or more C, N, O, and/or S.

Definitions:

As used herein, the term “halogen” shall mean chlorine, bromine, fluorine and iodine.

As used herein, the term “aliphatic” is intended to mean saturated and unsaturated, straight chain or branched aliphatic hydrocarbons. As will be appreciated by one skilled in the art, “aliphatic” is intended herein to include, but is not limited to, alkyl, alkenyl or alkynyl moieties.

As used herein, the term “alkyl” is intended to mean a straight chain or branched aliphatic group having from 1 to 12 carbon atoms, preferably 1-8 carbon atoms, and more preferably 1-6 carbon atoms. Other preferred alkyl groups have from 2 to 12 carbon atoms, preferably 2-8 carbon atoms and more preferably 2-6 carbon atoms. Preferred alkyl groups include, without limitation, methyl, ethyl, propyl, isopropyl, butyl, isobutyl, sec-butyl, tert-butyl, pentyl, hexyl and the like.

As used herein, the term “aryl” is intended to mean a mono-, bi-, tri, or polycyclic aromatic moiety, preferably a C₆-C₁₈ aromatic moiety, preferably comprising one to three aromatic rings. Preferably, the aryl group is a C₆-C₁₀ aryl group, more preferably a C₆ aryl group.

As used herein, the terms “heterocyclyl”, “heterocyclic” or “heterocycle” are intended to mean a group which is a mono-, bi-, or polycyclic structure having from about 3 to about 14 atoms, wherein one or more atoms are independently selected from the group consisting of N, O, and S. The ring structure may be saturated, unsaturated or partially unsaturated.

As used herein, the term “heteroaryl” is intended to mean a mono-, bi, tri- or polycyclic group having 5 to 18 ring atoms, preferably 5 to 14 ring atoms, more preferably 5, 6, 9, or 10 ring atoms; preferably having 6, 10, or 14 pi electrons shared in a cyclic array; and having, in addition to carbon atoms, between one or more heteroatoms selected from the group consisting of N, O, and S. The term “heteroaryl” is also intended to encompass the N-oxide derivative (or N-oxide derivatives, if the heteroaryl group contains more than one nitrogen such that more than one N-oxide derivative may be formed) of a nitrogen-containing heteroaryl group.

Representative of such compounds in accordance with the present invention are those shown below next to their respective designations used herein:

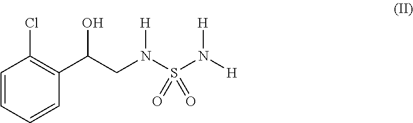

or a pharmaceutically accepted salt thereof;

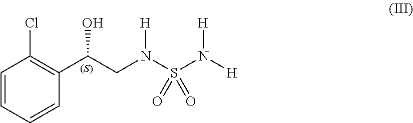

or a pharmaceutically accepted salt thereof;

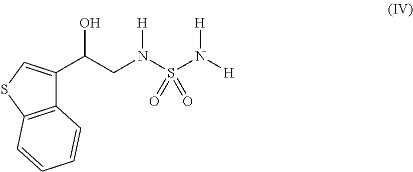

or a pharmaceutically accepted salt thereof.

As used herein, the terms “treat”, “treating” and “treatment” are intended, except where the context demands otherwise, to encompass administration to a patient of an effective dose of the novel hydroxyethyl sulfamides exhibiting neuroprotective action to mitigate or prevent neuronal cell death and damage from glutamate toxicity.

As used herein, an “effective dose” (or “effective amount”) is intended to mean a dose (or amount), which produces the effect of mitigation or prevention of neuronal cell death and damage from glutamate toxicity.

As used herein, a “subject” is any mammal suffering from glutamate toxicity, but most typically is a human subject. One skilled in the art can readily determine if a subject is suffering from glutamate toxicity or toxicity symptoms by performing a standard clinical or neurological assessment as is well known in the art.

As used herein, the notation “*” shall denote the presence of a stereogenic center.

Abbreviations used in the specification, particularly the Schemes and Examples, are as follows:

DCC=Dicyclohexyl Carbodiimide

DCE=Dichloroethane

DCM=Dichloromethane

DIPEA or DIEA Diisopropylethylamine

DMF=N,N-Dimethylformamide

DMSO=Dimethylsulfoxide

EDC=Ethylcarbodiimide

Et₃N or TEA=Triethylamine

Et₂O=Diethyl ether

EA or EtOAc=Ethyl acetate

EtOH=Ethanol

IPA=2-propanol

Hept=Heptane

HOBT=1-Hydroxybenzotriazole

M or MeOH=Methanol

NMR=Nuclear Magnetic Resonance

RT or rt=Room temperature

TFA=Trifluoroacetic Acid

THF=Tetrahydrofuran

TLC=Thin Layer Chromatography

As used herein, the term “composition” is intended to encompass a product comprising the specified ingredients in the specified amounts, as well as any product which results, directly or indirectly, from combinations of the specified ingredients in the specified amounts.

The phrase “pharmaceutically accepted” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The scientific plan for devising neuroprotective anticonvulsants in accordance with the present invention was to take existing compounds known to have the desired properties and to optimize their structure for improved efficacy, potency and safety. For this purpose, several known anticonvulsants were employed. Carisbamate is a new antiepileptic drug that is a derivative of felbamate, but with improved tolerability (Johannessen and Johannessen, 2008). With more than a dozen publications reporting the properties of carisbamate, this compound is a broad spectrum anticonvulsant (Keck et al., 2007) with neuroprotective properties (Deshpande et al., 2008). Unique analogues of carisbamate and other anti-seizure compounds (Smith-Swintosky and Reitz, 2004) have been devised with improved properties over the starting compounds.

Therefore, in accordance with the present invention novel neuroprotective compounds were devised by making unique derivatives of known anticonvulsants that involve the use of the hydroxyethyl sulfamide (HES) functionality. These compounds have unique core structures that reside in hydroxyethyl sulfamides and have exhibited neuroprotective properties on neurons derived from the nervous system. The neuroprotective action of the HES compounds were determined from assays that measure the viability and cell death of hippocampal neurons treated with glutamate, an excitotoxin known to be involved in many types of neurodegenerative disease. At concentrations of 30 to 100 μM, the novel HES compounds were shown to attenuate glutamate toxicity on cultured hippocampal neurons as measured by the fluorescent viability dye carboxyfluorescein diacetate (Petroski and Geller, 1994) and by propidium iodide (Sarafian et al., 2002), a cell fluorescent dye that does not penetrate live cells but does stain DNA in cells with damaged membranes or in dead cells. Potency and/or efficacy of the novel sulfamides exceed that of the neuroprotective properties of the reference anticonvulsant carisbamate.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (−)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods well known in the art.

In some embodiments, the present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in Design of Prodrugs, ed. H. Bundgaard, Elsevier, 1985.

In a preferred embodiment, the present invention provides pharmaceutical compositions comprising a compound of formula (I), and a compound of formula (I) in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of the principle ingredient. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 1 to about 1000 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

In some embodiments, the present invention illustratively provides liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinylpyrrolidone or gelatin.

To prepare the pharmaceutical compositions of this invention, a compound of formula (I) is intimately admixed with a pharmaceutical carrier according to conventional pharmaceutical compounding techniques, which carrier may take a wide variety of forms depending on the form of preparation desired for administration, e.g., i.v. sterile injectable formulations will be prepared using appropriate solubilizing agents. A unit dose would contain about 10 to about 300 mg of the active ingredient. The tablets contain some or all of the following inactive ingredients: lactose hydrous, pregelatinized starch, microcrystalline cellulose, sodium starch glycolate, magnesium stearate, purified water, carnauba wax, hydroxypropyl methylcellulose, titanium dioxide, polyethylene glycol, synthetic iron oxide, and polysorbate 80. One skilled in the art will recognize that oral tablets containing a compound of formula (I) may be similarly prepared and may contain similar inactive ingredients.

One skilled in the art will recognize that pharmaceutical compositions comprising more than one active ingredient may be similarly prepared according to known methods.

In an illustrative example of an embodiment of the present invention, the stereo-center on the compound of formula (I) is in the S-configuration. In another embodiment of the present invention, the stereo-center on the compound of formula (I) is in the R-configuration.

In some embodiments of the present invention the compound of formula (I) is present as an enantiomerically enriched mixture, wherein the % enantiomeric enrichment (% ee) is greater than about 75%, preferably greater than about 90%, more preferably greater than about 95%, most preferably greater than about 98%.

Compounds of formula (I) may be prepared according to the process outlined in Scheme I, below.

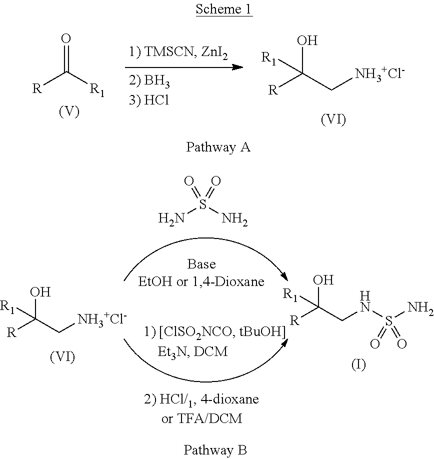

Accordingly, in this preferred embodiment, a suitably substituted compound of formula (V), a known compound or compound prepared by known methods, is reacted with trimethylsilyl cyanide, in the presence of a catalyst, like zinc iodide, in an organic solvent such as dichloromethane and the like, to give the trimethylsilyl ether cyanohydrin. This is then reduced in the presence of borane (as a complex with tetrahydrofuran, dimethylsulfide and the like), whose subsequent complex is decomposed with acid, such as hydrochloric acid in an organic solvent such as methanol ethanol, and the like, to yield the corresponding compound of formula (VI).

A compound of formula (VI) can then be converted into a hydroxyethyl sulfamide compound of formula (I) via multiple pathways.

Pathway A: Treatment of a compound of formula (VI) with sulfamide in the presence of a base, such as Et3N, N-methylmorpholine, and the like, in an organic solvent such as EtOH, dioxane and the like, at elevated temperatures such as between 50° C. and reflux, provides directly a hydroxyethyl sulfamide of formula (I).

Pathway B: A compound of formula (VI) can be treated with chlorosulfonyl-tert-butylcarbamate, formed in situ by the reaction of chlorosulfonylisocyanate and tert-butanol in an organic solvent like dichloromethane and the like to yield the tert-butylcarbamate of a compound of formula (VI). This can be treated with acid, such as hydrogen chloride, TFA, and the like in organic solvent such as dioxane, dichloromethane, and the like, to give a hydroxyethyl sulfamide of formula (I).

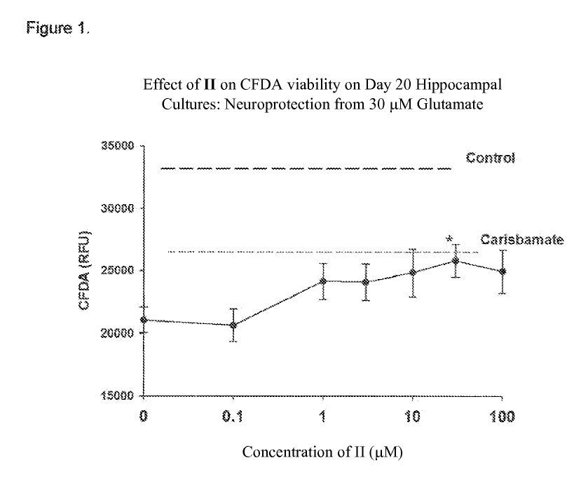

Pharmacology: Hydroxyethyl sulfamides attenuate glutamate-mediated neuronal viability. As an example, compound II, shown above, is shown to attenuate a decrease in carboxyfluorescein diacetate (CFDA) produced by acute treatment with glutamate in FIG. 1. For these studies, day 20 hippocampal cultures derived from E18 rats were treated for 5 min with 30 μM glutamate. Following removal of the glutamate, cultures were treated with various concentrations of compound II and the CFDA neuronal viability assay was conducted 24 hours after the initiation of treatment. The dotted line labeled “control” represents the mean CFDA value for control culture receiving no treatment. The dotted line labeled “carisbamate” represents the mean CFDA value for the reference compound carisbamate, a putative neuroprotective, anticonvulsant. For the CFDA measurements, RFU is the abbreviation for relative fluorescent units. The reference anticonvulsant compound carisbamate was tested at 100 μM, as lower concentrations were ineffective.

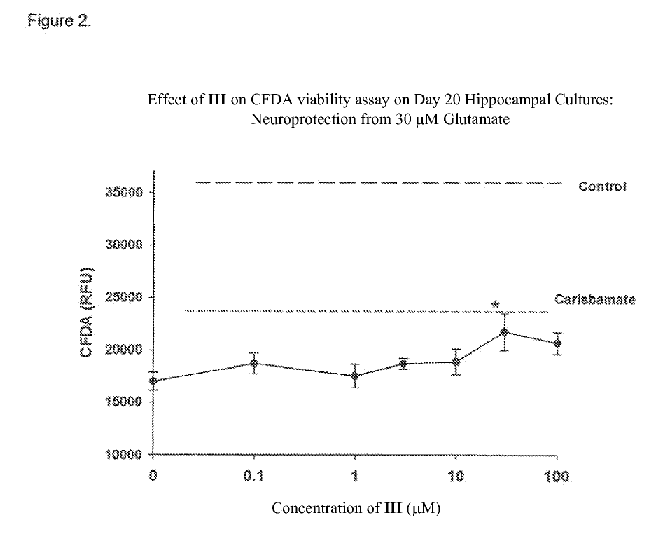

As another example of a hydroxyethyl sulfamide that attenuated the loss of glutamate-mediated decrease in neuronal viability, compound III is shown to be efficacious by the CFDA method in FIG. 2. The dotted line labeled “control” represents the mean CFDA value for control culture receiving no treatment. The dotted line labeled “carisbamate” represents the mean CFDA value for the reference compound carisbamate, a putative neuroprotective, anticonvulsant.

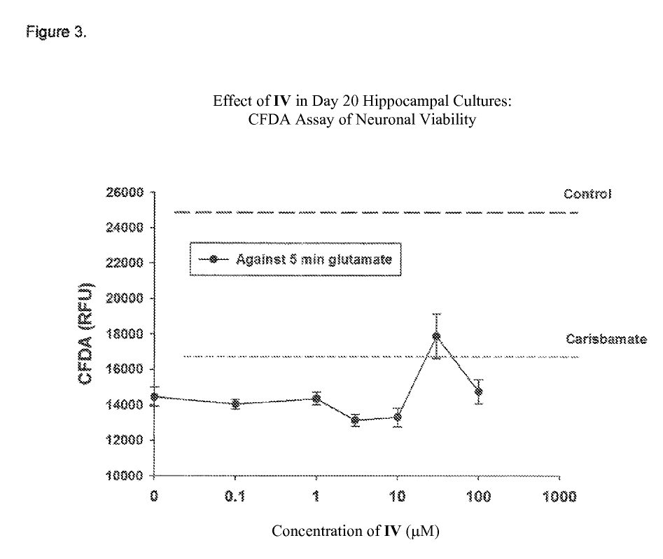

As a third example of a hydroxyethyl sulfamide that attenuated the loss of glutamate-mediated decrease in neuronal viability, compound IV is shown to be efficacious by the CFDA method in FIG. 3. Similar to previous examples, the dotted line labeled “control” represents CFDA values from control cultures and the dotted line labeled “carisbamate” represents values resulting from treatment with 100 μM Carisbamate, the reference anticonvulsant.

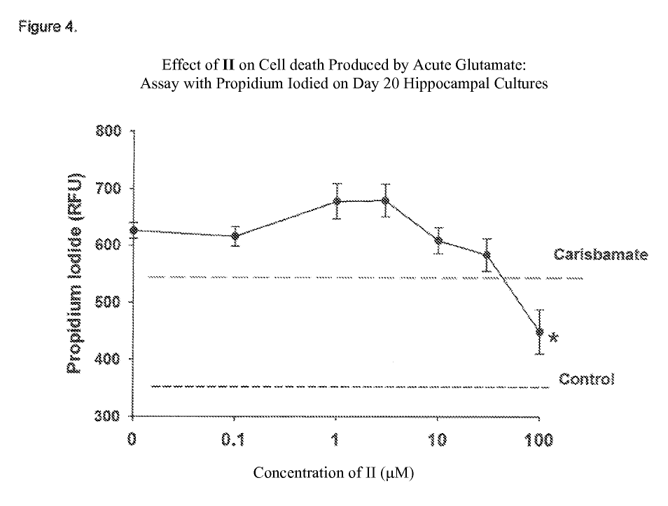

Hydroxyethyl sulfamides attenuate glutamate-mediated cell death: As an example of HES compounds that prevent cell death associated with glutamate, compound II is shown to attenuate an increase in propidium iodide (PI) produced by acute treatment with glutamate in FIG. 4. For these studies, day 20 hippocampal cultures derived from E18 rats were treated for 5 min with 30 μM glutamate. Following removal of the glutamate, cultures were treated with various concentrations of compound II and the propidium iodide method for measuring cell death was conducted 24 hours after the initiation of treatment. The dotted line labeled “control” represents the mean PI value for control culture receiving no treatment. The dotted line labeled “carisbamate” represents the mean PI value for the reference compound carisbamate, a putative neuroprotective anticonvulsant. The amount of carisbamate used was 100 μM and this concentration had the maximally observed efficacy for this compound. RFU is an abbreviation for relative fluorescent units. Day 15 is the number of days that the cultures were maintained in vitro before conducting the experiment.

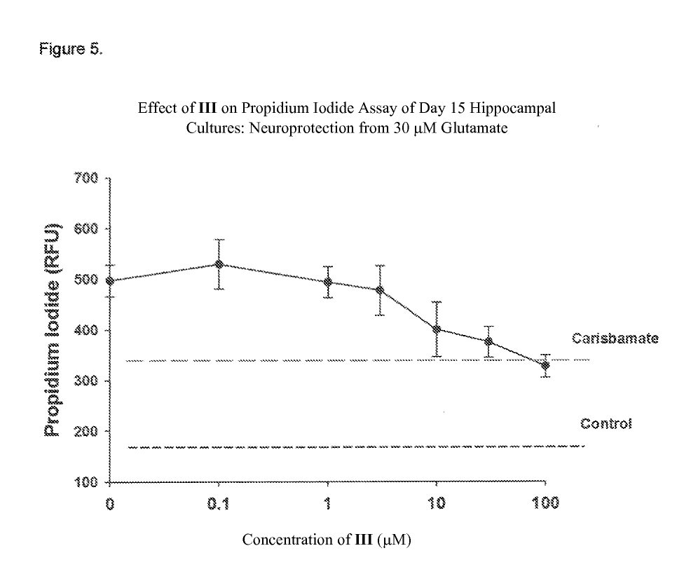

As another example of a hydroxyethyl sulfamide that attenuated the loss of glutamate-mediated increase in cell death, compound III is shown to be efficacious by the PI method in FIG. 5. The dotted line labeled “control” represents the mean PI value for control culture receiving no treatment. The dotted line labeled “carisbamate” represents the mean PI value for the reference compound carisbamate, a putative neuroprotective anticonvulsant.

As has been shown, the neuroprotective activity of the sulfamide derivates in accordance with the invention as described herein predicts a disease-modifying action in the treatment of diseases associated with excitotoxicity and accompanying oxidative stress that include epilepsy, Alzheimer's disease, and any neurodegenerative disease involving glutamate toxicity. The specific enabling conditions against the neurotoxin glutamate provide the evidence that sulfamide compounds would have a significant beneficial effect on brain neuron structure and survival that could alter the progression of these neurodegenerative diseases.

The phrase “pharmaceutically-acceptable carrier” as used herein means a pharmaceutically-acceptable material, composition or vehicle, such as a liquid or solid filler, diluent, excipient, manufacturing aid (e. g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other components and agents of the formulation and not deleterious to the patient after administration. In some illustratively examples, pharmaceutically-acceptable carriers can include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; any polyol, such as, but not limited to, glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; (19) ethyl alcohol; pH buffered solutions; polyesters, polycarbonates and/or polyanhydrides; and other nontoxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions of the invention.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. In some embodiments, a formulation of the present invention comprises an excipient selected from the group consisting of cyclodextrins, celluloses, liposomes, micelle forming agents, e. g., bile acids, and polymeric carriers, e. g., polyesters and polyanhydrides. In some embodiments, an aforementioned formulation renders orally bioavailable a compound of the present disclosure.

Methods of preparing these formulations or compositions include the step of bringing into association one or more compositions according to the present invention with the carrier and, optionally, one or more accessory ingredients. In general, the formulations are prepared by uniformly and mixing the compositions with liquid carriers, or finely divided solid carriers, or both, and then, if necessary, shaping the product. Formulations of the disclosure suitable for oral administration may be in the form of capsules, cachets, pills, tablets, lozenges, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) each containing an effective amount of a composition in accordance with the invention as an active ingredient. A controlled release formulation of the present invention may also be administered as a bolus, electuary or paste.

In solid dosage forms of the present invention for oral administration (capsules, tablets, pills, dragees, powders, granules, troches and the like), the composition provided by the invention is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as glycerol and paraffin; absorption accelerators, such as quaternary ammonium compounds and surfactants, such as poloxamer and sodium lauryl sulfate; wetting agents, such as, for example, cetyl alcohol, glycerol monostearate, and non-ionic surfactants; lubricants, such as talc, calcium stearate, magnesium stearate, solid polyethylene glycols, sodium lauryl sulfate, zinc stearate, sodium stearate, stearic acid; coloring agents; and controlled release agents such as crospovidone or ethyl cellulose. In the case of capsules, tablets and pills, the pharmaceutical compositions can also comprise buffering agents. Solid compositions of controlled release formulations can also be used as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as capsules, pills and granules, may optionally be scored or prepared with coatings and shells, such as enteric coatings and other coatings well known in the pharmaceutical-formulating art. In some embodiments, controlled release pharmaceutical compositions can be formulated so as to provide immediate, sustained, delayed, pulsed, biphasic release of the compositions provided by the invention using, for example, hydroxypropylmethyl cellulose in varying proportions to provide the desired release profile, other polymer matrices, liposomes and/or microspheres. The compositions may be prepared for immediate release, e.g., freeze-dried. In some embodiments, the compositions can be sterilized by, for example, filtration through a bacteria-retaining filter, or by incorporating sterilizing agents in the form of sterile solid compositions which can be dissolved in sterile water, or some other sterile injectable medium immediately before use. The compositions can also optionally contain opacifying agents and may be of a composition that they release the active ingredients preferentially in a certain portion of the gastrointestinal tract, or optionally, in a delayed manner. Examples of embedding compositions which can be used include polymeric substances and waxes. The compositions can also be in micro-encapsulated form, if appropriate, with one or more of the above described excipients.

The compositions of the invention intended for oral use can be prepared according to any method commonly known in the art. The pharmaceutical compositions of the present invention can contain one or more agents selected from the group consisting non-toxic pharmaceutically acceptable excipients which are suitable for the manufacture of tablets. Such excipients include, for example, an inert diluent such as lactose; granulating and disintegrating agents such as cornstarch; binding agents such as starch; and lubricating agents such as magnesium stearate. The tablets may be uncoated or they may be coated by known techniques for elegance or to delay release of the active ingredients. Formulations for oral use may also be presented as hard gelatin capsules wherein the active ingredient is mixed with an inert diluent and a controlled and/or immediate release matrix or coating.

Liquid dosage forms for oral administration of the compounds of the invention can include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active ingredients can be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an active ingredient of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Pharmaceutical compositions of the present invention can be suitable for parenteral administration comprising the active compound in a controlled release formulation in combination with one or more pharmaceutically-accepted sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

It will be appreciated that one skilled in the art can readily prepare pharmaceutical compositions comprising the compositions of the present invention, for example by using the principles set forth in Remington's Pharmaceutical Science, 18th Edition (Alphonso Gennaro, ed.), Mack Publishing Co., Easton, Pa., 1990 in conjunction with the teachings of the present disclosure of the invention herein.

The compositions of the present invention may be given enterally, illustratively orally, parenterally, topically, or rectally. They are of course provided in forms suitable for each administration route. For example, they can be administered in tablets or capsule form, by injection, inhalation, eye lotion, ointment, suppository, etc. administration by injection, infusion or inhalation; topical by lotion or ointment; and rectal by suppositories. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The compositions of the present invention may be administered to humans and other animals, including rats, mice, rabbits, dogs, cats, non-human primates, and monkeys for therapy by any suitable route of administration, including orally, nasally, as by, for example, a spray, rectally, parenterally, intracisternally and topically, as by powders, ointments or drops, including buccally and sublingually. Regardless of the route of administration selected, the compounds of the present invention, which may be used in a suitable hydrated form, and/or the pharmaceutical compositions of the present disclosure, are formulated into pharmaceutically-accepted dosage forms by conventional methods known to those of skill in the art.

In some embodiments a method is provided for treating glutamate toxicity in a patient comprising: administering to the patient a once daily composition comprising a single unit dose of a composition according to the present invention, formulated to provide a release profile wherein sufficient of the composition is delivered to the brain of the subject to enhance efficiency or reduce the side effects of glutamate toxicity for a number of hours after administration.

Administering a pharmaceutical composition comprising a controlled release formulation of the compositions in accordance with the invention also may reduce the frequency or severity of side effects.

The present invention also provides a kit comprising a formulation comprising of a composition in accordance with the invention and instructions for administering the same.

Additional embodiments, as well as features, benefits and advantages, of the present invention will be apparent to those skilled in the art, taking into account the foregoing description of preferred embodiments of the invention. It is therefore to be appreciated that the present invention is not to be construed as being in any way limited by the foregoing description of such preferred embodiments, but that various changes and modifications can be made to the invention as specifically described herein, and that all such changes and modifications are intended to be within the scope of the present invention.

### EXAMPLES

The examples provided below provide representative methods for preparing exemplary compounds of the present invention. The skilled practitioner will know how to substitute the appropriate reagents, starting materials and purification methods known to those skilled in the art, in order to prepare the compounds of the present invention.

¹H-NMR spectra were obtained on a Varian Mercury 300-MHz NMR. Purity (%) and mass spectral data were determined with a Waters Alliance 2695 HPLC/MS (Waters Symmetry C18, 4.6×75 mm, 3.5 μm) with a 2996 diode array detector from 210-400 nm.

### Example 1

### Synthesis of 2-(2-Chlorophenyl)-2-hydroxyethylsulfamide

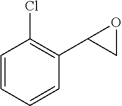

2-(2-Chloro-phenyl)-oxirane. 2-Chloro-benzaldehyde (5 mL, 44 mmol) was dissolved in dichloromethane (45 mL) and treated with trimethylsulfonium iodide (10.25 g, 49.28 mmol) and sodium hydroxide solution (50%, 44 g) subsequently. The mixture was stirred vigorously at room temperature for overnight. The organic phase was separated and concentrated to give a clear oil, which was stored in the refrigerator and used directly without further purification.

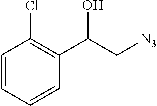

2-Azido-1-(2-chloro-phenyl)-ethanol. 2-(2-chloro-phenyl)-oxirane (250 mg, 1.62 mmol) and trimethylsilyl azide (0.33 mL, 2.42 mmol) were dissolved in tetrahydrofuran (8 mL). And the mixture was heated to 67° C., followed by addition of tetrabutylammonium fluoride (1M, 2.42 mL) dropwise. The mixture was stirred for 2 hr, and then concentrated. The residue was dissolved in ethyl acetate and washed with saturated aqueous sodium chloride. The organic layer was concentrated and purified by preparative thin-layer chromatography (2×2000 mm silica gel) developing with ethyl acetate/hexane (3:7) to give the product as a colorless oil (170 mg, 53%). ¹H NMR (CDCl₃) δ 7.70-7.60 (m, 1H), 7.40-7.20 (m, 3H), 5.40-5.20 (m, 1H), 3.60-3.50 (m, 1H), 3.40-3.30 (m, 1H), 3.05-2.95 (m, 1H).

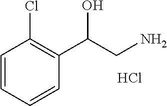

2-Amino-1-(2-chloro-phenyl)-ethanol hydrochloride salt. To a stirred solution of 2-azido-1-(2-chloro-phenyl)-ethanol (391 mg, 1.98 mmol) and triphenylphosphine (2.59 g, 9.89 mmol) in tetrahydrofuran (10 mL) was added water dropwise until it became cloudy. A few drops of tetrahydrofuran were added to make a clear solution, which was stirred at room temperature for a weekend. The reaction mixture was concentrated. The residue was partitioned between ethyl acetate and aqueous hydrochloric acid (2N). The aqueous phase was separated, extracted with ethyl acetate, and concentrated to give the product as a white solid (260 mg, 63%). ¹H NMR (CDCl₃) δ 7.75-7.65 (m, 1H), 7.45-7.30 (m, 3H), 5.35-5.25 (m, 1H), 3.35-3.20 (m, 1H), 2.95-2.85 (m, 1H).

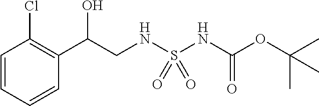

N-[2-(2-Chloro-phenyl)-2-hydroxy-1-carbamic tert-butyl ester-ethyl]-sulfamide. To a solution of chlorosulfonyl isocyanate (0.044 mL, 0.5 mmol) in dichloromethane (1 mL) at 3° C. was added tert-butanol (0.047 mL, 0.5 mmol). After 25 minutes, pyridine (0.089 mL, 1.09 mmol) was added and the resulting mixture was stirred for 40 minutes during which time a precipitate formed. This slurry was added via pipette to a mixture of 2-amino-1-(2-chloro-phenyl)-ethanol hydrochloride salt (104 mg, 0.5 mmol) and triethylamine (0.17 mL, 1.25 mmol) in dichloromethane (1 mL) at 3° C. The resulting mixture was allowed to warm to ambient temperature over 4 hours. The reaction mixture was diluted with dichloromethane (5 mL) and washed with aqueous potassium hydrogen sulfate (3 mL, 10%) followed by saturated aqueous sodium chloride (5 mL). The organic layer was concentrated at reduce pressure. The residue was purified by preparative thin-layer chromatography (2×2000 mm silica gel) developing with ethyl acetate/hexane (1:1) to give the product as a white solid (118.2 mg, 68%). ¹H NMR (CDCl₃) δ 7.68-7.58 (m, 1H), 7.40-7.18 (m, 3H), 5.30-5.18 (m, 1H), 3.50-3.30 (m, 1H), 3.16-3.00 (m, 1H), 1.50 (s, 9H). LC-MS for C₁₃H₁₉ClN₂O₅S: t=4.64 min. MS(ES⁺)=372.95 (M+Na)⁺.

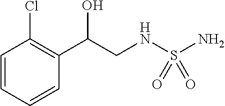

2-(2-Chlorophenyl)-2-hydroxyethylsulfamide. N-[2-(2-Chloro-phenyl)-2-hydroxy-1-carbamic tert-butyl ester-ethyl]-sulfamide (118.2 mg, 0.34 mmol) was treated with trifluoroacetic acid (5 mL, 20% in dichloromethane), and the resulting solution was stirred for 1 hours. The reaction mixture was concentrated at reduced pressure and the resulting solid was purified by preparative thin-layer chromatography (1×2000 mm silica gel) developing with ethyl acetate/hexane (1:1) to give the product as a white solid (78.8 mg, 92%). ¹H NMR (DMSO-d₆) δ 7.62-7.55 (m, 1H), 7.42-7.20 (m, 3H,), 6.45 (s, 2H), 6.35 (t, J=7.1 Hz, 1H), 5.60 (d, J=4.7 Hz, 1H), 5.08-4.96 (m, 1H), 3.20-3.05 (m, 1H), 2.90-2.80 (m, 1H); LC-MS for C₈H₁₁ClN₂O₃S: t=2.94 min. MS(ES⁺)=233.00 (M−H₂O+H)⁺.

### Example 2

### Synthesis of 2-(3-Chlorophenyl)-2-hydroxyethylsulfamide

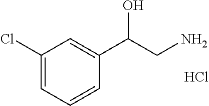

2-Amino-1-(3-chloro-phenyl)-ethanol hydrochloride salt. To a stirring solution of 3-Chloro-benzaldehyde (1.66 g, 11.81 mmol) and trimethylsilyl cyanide (1.77 mL, 12.99 mmol) in dichloromethane (12 mL) at room temperature under nitrogen was added a few crystals of zinc iodide. The mixture was stirred at this temperature for 20 hours and concentrated. The residue was dissolved in borane in tetrahydrofuran (1M, 26 mL) and heated to reflux for 5 hours. The mixture was cooled to room temperature and concentrated. The syrup was dissolved in methanol (30 mL), treated with hydrogen chloride in dioxane (4M, 13 mL), and heated to reflux for 2 hours, and then concentrated. The solid obtained was dissolved in water and extracted twice with diethyl ether. The aqueous solution was concentrated, and dried in vacuum oven to give a white solid (2.1 g, 85%).

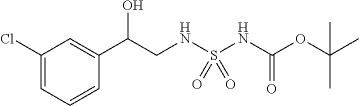

N-[2-(3-Chloro-phenyl)-2-hydroxy-1-carbamic tert-butyl ester-ethyl]-sulfamide. To a solution of chlorosulfonyl isocyanate (0.38 mL, 4.32 mmol) in dichloromethane (5 mL) at 3° C. was added tert-butanol (0.41 mL, 4.32 mmol). After 25 minutes, pyridine (0.77 mL, 9.50 mmol) was added and the resulting mixture was stirred for 40 minutes during which time a precipitate formed. This slurry was added via pipette to a mixture of 2-amino-1-(3-chloro-phenyl)-ethanol hydrochloride salt (900 mg, 4.32 mmol) and triethylamine (1.5 mL, 10.8 mmol) in dichloromethane (5 mL) at 3° C. The resulting mixture was allowed to warm to ambient temperature over 4 hours. The reaction mixture was diluted with dichloromethane (20 mL) and washed with aqueous potassium hydrogen sulfate (5 mL, 10%) followed by saturated aqueous sodium chloride (5 mL). The organic layer was concentrated at reduce pressure. The residue was purified by column chromatography (80 g silica gel cartridge) eluting with ethyl acetate/hexane (10%-30%) to give the product as a white solid (1.01 g, 67%), which was used in the next step directly.

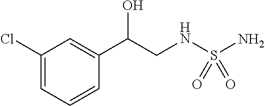

2-(3-Chlorophenyl)-2-hydroxyethylsulfamide. N-[2-(3-Chloro-phenyl)-2-hydroxy-1-carbamic tert-butyl ester-ethyl]-sulfamide prepared above was treated with trifluoroacetic acid (5 mL, 20% in dichloromethane), and the resulting solution was stirred for 1 hour. The reaction mixture was concentrated at reduced pressure and the resulting solid was purified by preparative thin-layer chromatography developing with ethyl acetate/hexane (1:1) to give the product as a white solid (420 mg, 58%). ¹H NMR (DMSO-d₆) δ 7.42-7.25 (m, 4H), 6.54 (s, 2H), 6.35 (t, J=6.0 Hz, 1H), 5.56 (d, J=4.5 Hz, 1H), 4.75-4.64 (m, 1H), 3.10-2.95 (m, 2H); LC-MS for C₈H₁₁ClN₂O₃S: t=2.94 min. MS(ES⁺)=233.00 (M−H₂O+H)⁺.

### Example 3

### Synthesis of 2-(thiophen-3-yl)-2-hydroxyethylsulfamide

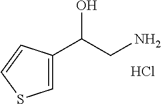

2-Amino-1-thiophen-3-yl-ethanol hydrochloride salt. To a stirring solution of thiophene-3-carbaldehyde (1.0 g, 8.9 mmol) and trimethylsilyl cyanide (1.33 mL, 9.81 mmol) in dichloromethane (10 mL) at room temperature under nitrogen was added a few crystals of zinc iodide. The mixture was stirred at this temperature for 20 hours and concentrated. The residue was dissolved in borane in tetrahydrofuran (1M, 19.6 mL) and heated to reflux for 5 hours. The mixture was cooled to room temperature and concentrated. The syrup was dissolved in methanol (25 mL), treated with hydrogen chloride in dioxane (4M, 9.8 mL), and heated to reflux for 2 hours, and then concentrated. The solid obtained was dissolved in aqueous hydrochloric acid (2N, 10 mL) and extracted twice with diethyl ether. The aqueous solution was concentrated, and dried in vacuum oven to give a yellow solid (1.5 g, 94%).

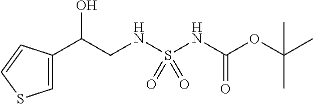

N-[(1-thiophen-3-yl)-2-hydroxy-1-carbamic tert-butyl ester-ethyl]-sulfamide. To a solution of chlorosulfonyl isocyanate (0.74 mL, 8.35 mmol) in dichloromethane (10 mL) at 3° C. was added tert-butanol (0.79 mL, 8.35 mmol). After 25 minutes, pyridine (1.48 mL, 18.37 mmol) was added and the resulting mixture was stirred for 40 minutes during which time a precipitate formed. This slurry was added via pipette to a mixture of 2-amino-1-thiophen-3-yl-ethanol hydrochloride salt (1.5 g, 8.35 mmol) and triethylamine (2.91 mL, 20.88 mmol) in dichloromethane (10 mL) at 3° C. The resulting mixture was allowed to warm to ambient temperature over 4 hours. The reaction mixture was diluted with dichloromethane (20 mL) and washed with cold hydrochloric acid (1M, 10 mL) followed by saturated aqueous sodium chloride (10 mL). The organic layer was concentrated at reduce pressure. The residue was purified by column chromatography (80 g silica gel cartridge) eluting with ethyl acetate/hexane (10%-30%) to give the product as a white solid (1.2 g, 44%). LC-MS for C₁₁H₁₈N₂O₅S₂: t=4.05 min. MS(ES⁺)=344.89 (M+Na)⁺.

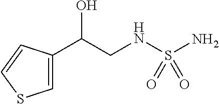

2-(thiophen-3-yl)-2-hydroxyethylsulfamide. N-[(1-thiophen-3-yl)-2-hydroxy-1-carbamic tert-butyl ester-ethyl]-sulfamide (1.2 g, 3.72 mmol) was dissolved in methanol (8 mL) and treated with hydrogen chloride (4 M in dioxane, 8 mL). The resulting solution was stirred for 2 hours. The reaction mixture was concentrated at reduced pressure and the resulting solid was purified by preparative thin-layer chromatography developing with ethyl acetate/hexane (1:1) to give the product as a white solid (200 mg, 17%). ¹H NMR (DMSO-d₆) δ 7.50-7.44 (m, 1H), 7.37-7.30 (m, 1H), 7.14-7.06 (m, 1H), 6.54 (s, 2H), 6.28 (t, J=7.0 Hz, 1H), 5.40 (d, J=4.8 Hz, 1H), 4.80-4.70 (m, 1H), 3.20-2.95 (m, 2H); LC-MS for C₆H₁₀N₂O₃S₂: t=2.03 min. MS(ES⁺)=205.0 (M−H₂O+H)⁺.

### Example 4

### Synthesis of 2-(benzothiophen-2-yl)-2-hydroxyethylsulfamide

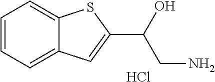

2-Amino-1-benzo[b]thiophen-2-yl-ethanol hydrochloride salt. To a stirring solution of benzo[b]thiophene-2-carbaldehyde (1.0 g, 6.16 mmol) and trimethylsilyl cyanide (0.92 mL, 6.78 mmol) in dichloromethane (8 mL) at room temperature under nitrogen was added a few crystals of zinc iodide. The mixture was stirred at this temperature for 20 hours and concentrated. The residue was dissolved in borane in tetrahydrofuran (1M, 13.6 mL) and heated to reflux for 5 hours. The mixture was cooled to room temperature and concentrated. The syrup was dissolved in methanol (25 mL), treated with hydrogen chloride (4M in dioxane, 6.8 mL), and heated to reflux for 2 hours, and then concentrated. The solid obtained was dissolved in hydrochloric acid (2N, 10 mL) and extracted twice with diethyl ether. The aqueous solution was concentrated, crystallized from methanol/ether, and dried in vacuum oven to give a yellow (770 mg, 54%).

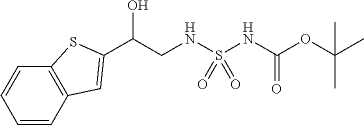

N-[2-(1-benzo[b]thiophen-2-yl-)-2-hydroxy-1-carbamic tert-butyl ester-ethyl]-sulfamide. To a solution of chlorosulfonyl isocyanate (0.30 mL, 3.35 mmol) in dichloromethane (5 mL) at 3° C. was added tert-butanol (0.32 mL, 3.35 mmol). After 25 minutes, pyridine (0.60 mL, 7.37 mmol) was added and the resulting mixture was stirred for 40 minutes during which time a precipitate formed. This slurry was added via pipette to a mixture of 2-amino-1-benzo[b]thiophen-2-yl-ethanol hydrochloride salt (770 mg, 3.35 mmol) and triethylamine (1.17 mL, 8.38 mmol) in dichloromethane (5 mL) at 3° C. The resulting mixture was allowed to warm to ambient temperature over 4 hours. The reaction was quenched with methanol (5 mL) and concentrated. The residue was suspended in ethyl acetate, washed with cold hydrochloric acid (1M, 10 mL) followed by saturated aqueous sodium chloride (10 mL). The organic layer was concentrated at reduce pressure. The residue was purified by column chromatography (80 g silica gel cartridge) eluting with ethyl acetate/hexane (10%-30%) to give the product as a white solid, which was used directly in the next step. LC-MS for C₁₅H₂ON₂O₅S₂: t=4.93 min. MS(ES⁺)=394.85 (M+Na)⁺.

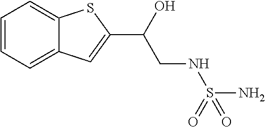

2-(benzothiophen-2-yl)-2-hydroxyethylsulfamide. N-[2-(1-benzo[b]thiophen-2-yl-)-2-hydroxy-1-carbamic tert-butyl ester-ethyl]-sulfamide prepared above was dissolved in methanol (8 mL) and treated with hydrogen chloride (4 M in dioxane, 8 mL). The resulting solution was stirred for 2 hours. The reaction mixture was concentrated at reduced pressure and the resulting solid was purified by preparative thin-layer chromatography developing with ethyl acetate/hexane (1:1) to give the product as a white solid (326.6 mg, 36% for two steps). ¹H NMR (DMSO-d₆) δ 7.96-7.88 (m, 1H), 7.82-7.75 (m, 1H), 7.40-7.26 (m, 3H), 6.60 (s, 2H), 6.51 (t, J=6.0 Hz, 1H), 6.01 (d, J=5.1 Hz, 1H), 5.10-4.96 (m, 1H), 3.25-3.10 (m, 2H); LC-MS for C₁₀H₁₂N₂O₃S₂: t=3.48 min. MS(ES⁺)=254.99 (M−H₂O+H)⁺.

### Example 5

### Synthesis of 2-(2,5-dichlorophenyl)-2-hydroxyethylsulfamide

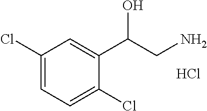

2-Amino-1-(2,5-dichloro-phenyl)-ethanol hydrochloride salt. To a stirring solution of 2,5-dichloro-benzaldehyde (1.0 g, 5.60 mmol) and trymethylsilyl cyaninde (0.84 mL, 6.16 mmol) in dichloromethane (6 mL) at room temperature under nitrogen was added a few crystals of zinc iodide. The mixture was stirred at this temperature for 20 hours and concentrated. The residue was dissolved in borane in tetrahydrofuran (1M, 12.32 mL) and heated to reflux for 5 hours. The mixture was cooled to room temperature and concentrated. The syrup was dissolved in methanol (15 mL), treated with hydrogen chloride (4M in dioxane, 6.2 mL), and heated to reflux for 2 hours, and then concentrated. The solid obtained was dissolved in water and extracted twice with diethyl ether. The aqueous solution was concentrated, and dried in vacuum oven to give a white solid (830 mg, 60%).

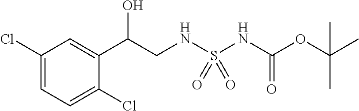

N-[2-(2,5-Dichloro-phenyl)-2-hydroxy-1-carbamic tert-butyl ester-ethyl]-sulfamide. To a solution of chlorosulfonyl isocyanate (0.30 mL, 3.42 mmol) in dichloromethane (4 mL) at 3° C. was added tert-butanol (0.32 mL, 3.42 mmol). After 25 minutes, pyridine (0.61 mL, 7.52 mmol) was added and the resulting mixture was stirred for 40 minutes during which time a precipitate formed. This slurry was added via pipette to a mixture of 2-amino-1-(2,5-dichloro-phenyl)-ethanol hydrochloride salt (830 mg, 3.42 mmol) and triethylamine (1.19 mL, 8.55 mmol) in dichloromethane (5 mL) at 3° C. The resulting mixture was allowed to warm to ambient temperature over 4 hours. The reaction was quenched with methanol (5 mL) and concentrated. The residue was suspended in ethyl acetate, washed with aqueous potassium hydrogen sulfate (5 mL, 10%) followed by saturated aqueous sodium chloride (5 mL). The organic layer was concentrated at reduce pressure. The residue was purified by column chromatography (80 g silica gel cartridge) eluting with ethyl acetate/hexane (10%-30%) to give the product as a white solid, which was used in the next step directly.

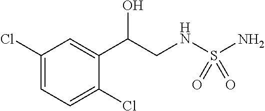

2-(2,5-dichlorophenyl)-2-hydroxyethylsulfamide. N-[2-(2,5-Dichloro-phenyl)-2-hydroxy-1-carbamic tert-butyl ester-ethyl]-sulfamide prepared above was dissolved in methanol (8 mL) and treated with hydrogen chloride (4 M in dioxane, 8 mL). The resulting solution was stirred for 2 hours. The reaction mixture was concentrated at reduced pressure and the resulting solid was triturated with hot dichloromethane to give the product as a white solid (234 mg, 36% for two steps). ¹H NMR (DMSO-d₆) δ 7.57 (d, J=2.7 Hz, 1H), 7.48-7.35 (m, 2H,), 6.50 (s, 2H), 6.35 (t, J=6.9 Hz, 1H), 5.77 (d, J=4.8 Hz, 1H), 5.08-4.95 (m, 1H), 3.24-3.12 (m, 1H), 2.96-2.86 (m, 1H); LC-MS for C₈H₁₀Cl₂N₂O₃S: t=3.59 min. MS(ES⁺)=266.93 (M−H₂O+H)⁺.

### Example 6

### Synthesis of 2-(2-fluorophenyl)-2-hydroxyethylsulfamide

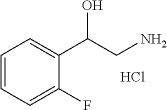

2-Amino-1-(2-fluoro-phenyl)-ethanol hydrochloride salt. To a stirring solution of 2-fluoro-benzaldehyde (1.0 g, 7.82 mmol) and trimethylsilyl cyanide (1.17 mL, 8.60 mmol) in dichloromethane (10 mL) at room temperature under nitrogen was added a few crystals of zinc iodide. The mixture was stirred at this temperature for 20 hours and concentrated. The residue was dissolved in borane in tetrahydrofuran (1M, 17.2 mL) and heated to reflux for 5 hours. The mixture was cooled to room temperature and concentrated. The syrup was dissolved in methanol (17 mL), treated with hydrogen chloride (4M in dioxane, 8.6 mL), and heated to reflux for 2 hours, and then concentrated. The solid obtained was dissolved in water and extracted twice with diethyl ether. The aqueous solution was concentrated, and dried in vacuum oven to give a white solid (1.22 g, 79%).

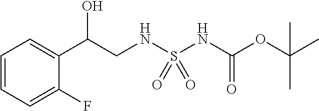

N-[2-(2-Fluoro-phenyl)-2-hydroxy-1-carbamic tert-butyl ester-ethyl]-sulfamide. To a solution of chlorosulfonyl isocyanate (0.45 mL, 5.22 mmol) in dichloromethane (10 mL) at 3° C. was added tert-butanol (0.50 mL, 5.22 mmol). After 25 minutes, pyridine (0.93 mL, 11.48 mmol) was added and the resulting mixture was stirred for 40 minutes during which time a precipitate formed. This slurry was added via pipette to a mixture of 2-amino-1-(2-fluoro-phenyl)-ethanol hydrochloride salt (1.09 g, 5.22 mmol) and triethylamine (1.82 mL, 13.05 mmol) in dichloromethane (10 mL) at 3° C. The resulting mixture was allowed to warm to ambient temperature over 4 hours. The reaction was quenched with methanol (5 mL) and concentrated. The residue was suspended in ethyl acetate, washed with aqueous potassium hydrogen sulfate (5 mL, 10%) followed by saturated aqueous sodium chloride (5 mL). The organic layer was concentrated at reduce pressure. The residue was purified by column chromatography (80 g silica gel cartridge) eluting with ethyl acetate/hexane (10%-40%) to give the product as a sticky oil, which was used in the next step directly. ¹H NMR (CDCl₃) δ 7.56-7.46 (m, 1H), 7.30-7.20 (m, 1H), 7.20-7.10 (m, 1H), 7.03-6.96 (m, 1H), 6.06 (br, 1H), 5.18 (dd, J=8.4, 3.0 Hz, 1H), 3.50-3.35 (m, 1H), 3.30-3.16 (m, 1H), 1.50 (s, 9H). LC-MS for C₁₃H₁₉ClN₂O₅S: t=4.33 min. MS(ES⁺)=356.8 (M+Na)⁺.

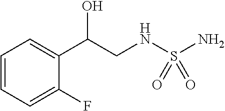

2-(2-fluorophenyl)-2-hydroxyethylsulfamide. N-[2-(2-Fluoro-phenyl)-2-hydroxy-1-carbamic tert-butyl ester-ethyl]-sulfamide prepared above was dissolved in methanol (8 mL) and treated with hydrogen chloride (4 M in dioxane, 8 mL). The resulting solution was stirred for 10 hours. The reaction mixture was concentrated at reduced pressure and the resulting solid was purified by column chromatography (40 g silica gel cartridge) eluting with ethyl acetate/hexane (10%-50%) to give the product as a white solid (234 mg, 26% for two steps). ¹H NMR (DMSO-d₆) δ 7.55-7.45 (m, 1H), 7.40-7.26 (m, 1H), 7.26-7.10 (m, 2H), 6.51 (s, 2H), 6.40 (t, J=5.7 Hz, 1H), 5.55 (d, J=4.8 Hz, 1H), 5.02-4.92 (m, 1H), 3.20-3.06 (m, 1H), 3.06-2.90 (m, 1H); LC-MS for C₈H₁₁FN₂O₃S: t=2.47 min. MS(ES⁺)=217.05 (M−H₂O+H)⁺.

### Example 7

### Synthesis of 2-(benzothiophen-3-yl)-2-hydroxyethylsulfamide

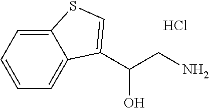

2-Amino-1-benzo[b]thiophen-3-yl-ethanol hydrochloride salt. To a stirring solution of thiophene-3-carbaldehyde (3.0 g, 17.94 mmol) and trimethylsilyl cyanide (2.68 mL, 19.73 mmol) in dichloromethane (20 mL) at room temperature under nitrogen was added a few crystals of zinc iodide. The mixture was stirred at this temperature for 20 hours and concentrated. The residue was dissolved in borane in tetrahydrofuran (1M, 40 mL) and heated to reflux for 5 hours. The mixture was cooled to room temperature and concentrated. The syrup was dissolved in methanol (60 mL), treated with hydrogen chloride (4M in dioxane, 20 mL), and heated to reflux for 2 hours, and then concentrated. The solid obtained was dissolved in aqueous hydrochloric acid (2N, 10 mL) and extracted twice with diethyl ether. The aqueous solution was concentrated, crystallized from methanol/ethyl acetate, and dried in vacuum oven to give a yellow solid (2.4 g, 58%).

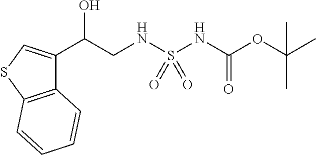

N-[2-(1-benzo[b]thiophen-3-yl-)-2-hydroxy-1-carbamic tert-butyl ester-ethyl]-sulfamide. To a solution of chlorosulfonyl isocyanate (0.39 mL, 4.35 mmol) in dichloromethane (10 mL) at 3° C. was added tert-butanol (0.41 mL, 4.35 mmol). After 25 minutes, pyridine (0.77 mL, 9.57 mmol) was added and the resulting mixture was stirred for 40 minutes during which time a precipitate formed. This slurry was added via pipette to a mixture of 2-amino-1-benzo[b]thiophen-3-yl-ethanol hydrochloride salt (1.0 g, 4.35 mmol) and triethylamine (1.52 mL, 10.88 mmol) in dichloromethane (10 mL) at 3° C. The resulting mixture was allowed to warm to ambient temperature over 4 hours. The reaction was quenched with methanol (5 mL) and concentrated. The residue was suspended in ethyl acetate, washed with aqueous potassium hydrogen sulfate (5 mL, 10%) followed by saturated aqueous sodium chloride (5 mL). The organic layer was concentrated at reduce pressure. The residue was purified by column chromatography (80 g silica gel cartridge) eluting with ethyl acetate/hexane (10%-40%) to give the product as a sticky oil, which was solidified on standing (1.18 g, 73%). ¹H NMR (CDCl₃) δ 7.94-7.80 (m, 2H), 7.52 (s, 1H), 7.46-7.32 (m, 2H), 5.70 (br, 1H), 5.40-5.28 (m, 1H), 3.68-3.50 (m, 1H), 3.44-3.28 (m, 1H), 1.50 (s, 9H). LC-MS for C₁₅H₂₀N₂O₅S₂: t=4.92 min. MS(ES⁺)=394.98 (M+Na)⁺.

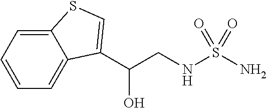

2-(benzothiophen-3-yl)-2-hydroxyethylsulfamide. N-[2-(1-benzo[b]thiophen-3-yl-)-2-hydroxy-1-carbamic tert-butyl ester-ethyl]-sulfamide (150 mg, 0.40 mmol) was treated with hydrogen chloride (2 M in ether, 2 mL). The resulting solution was stirred for 23 hours. The reaction mixture was concentrated at reduced pressure and the resulting solid was purified by preparative thin-layer chromatography developing with ethyl acetate/hexane (1:1) to give the desired product as a light yellow solid (8 mg, 7%). ¹H NMR (DMSO-d₆) δ 8.02-7.90 (m, 1H), 7.60 (s, 1H), 7.45-7.30 (m, 2H,), 6.58 (s, 2H), 6.51 (dd, J=6.6, 6.0 Hz, 1H), 5.59 (d, J=4.8 Hz, 1H), 5.15-5.05 (m, 1H), 3.35-3.22 (m, 1H), 3.20-3.05 (m, 1H); LC-MS for C₁₀H₁₂N₂O₃S₂: t=3.39 min. MS(ES⁺)=(M−H₂O+H)₊ 254.99.

### Example 8

### Synthesis of 2-(2-chlorophenyl)-2-methyl-2-hydroxyethylsulfamide

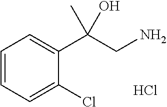

1-Amino-2-(2-chloro-phenyl)-propan-2-ol hydrochloride salt. To a stirring solution of 1-(2-chloro-phenyl)-ethanone (3.08 mL, 22.92 mmol) and trimethyl silyl cyanide (3.68 mL, 27.50 mmol) in dichloromethane (50 mL) at room temperature under nitrogen was added titanium tetrachloride (1M, 4.60 mL). The mixture was stirred at this temperature for 2 weeks and concentrated. The residue was purified on silica gel with a gradient of ethyl acetate:hexanes from 0:1 to 1:9 to provide 2-(2-chloro-phenyl)-2-hydroxy-propionitrile as a light yellow green oil (3.27 g, 78%). The oil (1.04 g, 5.73 mmol) was dissolved in borane in tetrahydrofuran (1M, 12.6 mL) and heated to reflux for 5 hours. The mixture was cooled to room temperature and concentrated. The syrup was dissolved in methanol (12.6 mL), treated with aqueous hydrochloric acid (2M, 12.6 mL), and heated to reflux for 2 hours, and then concentrated. The solid obtained was triturated with diethyl ether, and dried in vacuum oven to give a white solid (933.6 mg, 73%).

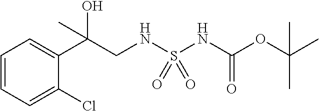

N-[2-(2-Chloro-phenyl)-2-hydroxy-1-carbamic acid tert-butyl ester propyl]-sulfamide. To a solution of chlorosulfonyl isocyanate (0.38 mL, 4.32 mmol) in dichloromethane (9 mL) at 3° C. was added tert-butanol (0.41 mL, 4.32 mmol). After 25 minutes, pyridine (0.77 mL, 9.50 mmol) was added and the resulting mixture was stirred for 40 minutes during which time a precipitate formed. This slurry was added via pipette to a mixture of 1-amino-2-(2-chloro-phenyl)-propan-2-ol hydrochloride salt (933.6 mg, 4.2 mmol) and triethylamine (1.5 mL, 10.8 mmol) in dichloromethane (9 mL) at 3° C. The resulting mixture was allowed to warm to ambient temperature over 4 hours. The reaction was quenched with methanol (5 mL) and concentrated. The residue was suspended in ethyl acetate, washed with aqueous potassium hydrogen sulfate (5 mL, 10%) followed by saturated aqueous sodium chloride (5 mL). The organic layer was concentrated at reduce pressure. The residue was purified by column chromatography (80 g silica gel cartridge) eluting with ethyl acetate/hexane (10%-30%) to give the product as a white solid, which was used in the next step directly.

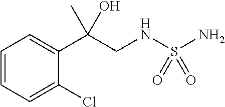

2-(2-chlorophenyl)-2-methyl-2-hydroxyethylsulfamide. N-[2-(2-Chloro-phenyl)-2-hydroxy-1-carbamic acid tert-butyl ester propyl]-sulfamide prepared above was dissolved in methanol (8 mL) and treated with hydrogen chloride (4 M in dioxane, 8 mL). The resulting solution was stirred for 4 hours. The reaction mixture was concentrated at reduced pressure and the resulting solid was purified by column chromatography (40 g silica gel cartridge) eluting with ethyl acetate/hexane (10%-50%) to give the product as a clear oil (1.0 g, 77% for two steps). ¹H NMR (DMSO-d₆) δ 7.83-7.79 (m, 1H), 7.40-7.24 (m, 3H), 6.52 (s, 2H), 5.85 (t, J=6.3 Hz, 1H), 5.42 (s, 1H), 3.48 (dd, J=12.3, 6.3 Hz 1H), 3.36 (dd, J=12.6, 6.6 Hz 1H), 1.61 (s, 3H). LC-MS for C₉H₁₃ClN₂O₃S: t=3.34 min. MS(ES⁺)=247.01 (M−H₂O+H)⁺.

### Example 9

### Synthesis of 3-(Benzo[b]thiophen-3-yl)-3-hydroxy-azetidine-1-sulfamide

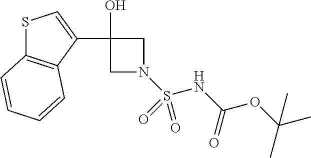

3-Benzo[b]thiophen-3-yl-3-hydroxy-azetidine-1-carboxylic acid tert-butyl ester. To a stirring solution of 3-Bromo-benzo[b]thiophene (1 g, 4.7 mmol) in tetrahydrofuran (15 mL) at 0° C. under nitrogen was added a solution of isopropylmagnesium chloride (2.6 mL, 5.17 mmol, 2.0 M in tetrahydrofuran). After 10 minutes, the reaction mixture was warmed to ambient temperature then warmed to 35° C. for one hour. The reaction mixture was cooled to 0° C. and 3-oxo-azetidine-1-carboxylic acid tert-butyl ester (1.04 g, 6.1 mmol) was added in one portion. After 30 minutes, the reaction was quenched with water (2 mL) and was allowed to warm to ambient temperature. After one hour at ambient temperature, the reaction mixture was poured into saturated aqueous ammonium chloride (35 mL) and the mixture was extracted with ethyl acetate (2×25 mL). The organic layers were combined and concentrated at reduced pressure. The resulting crude product was purified by column chromatography through a silica gel cartridge (12 g) eluting with ethyl acetate/hexane to give the product as a white solid (300 mg, 21%). ¹H NMR (CDCl₃) δ 7.83 (m, 1H,), 7.74 (m, 1H), 7.20-7.08 (m, 3H), 4.35 (d, J=8.4 Hz, 2H), 4.05 (d, J=8.4 Hz, 2H), 2.92 (s, 1H), 1.44 (m, 9H). MS(ES⁺)=306(MH)⁺.

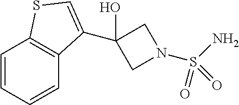

3-Benzo[b]thiophen-3-yl-3-hydroxy-azetidine-1-sulfonamide. To a stirring solution of 3-Benzo[b]thiophen-3-yl-3-hydroxy-azetidine-1-carboxylic acid tert-butyl ester (300 mg, 0.98 mmol) in ethyl acetate (2 mL) was added hydrogen chloride (4 mL, 4N in 1,4-dioxane) and the resulting solution was stirred for 3 hours during which time a precipitate formed. The reaction mixture was concentrated at reduced pressure to give the crude product, 3-benzo[b]thiophen-3-yl-azetidin-3-ol hydrochloride, which was used in the next step without purification. To a solution of chlorosulfonyl isocyanate (0.025 mL, 0.28 mmol) in dichloromethane (0.5 mL) at 3° C. was added tert-butanol (0.027 mL, 0.28 mmol). After 25 minutes, pyridine (0.050 mL, 0.62 mmol) was added and the resulting mixture was stirred for 40 minutes during which time a precipitate formed. This slurry was added via pipette to a solution of crude 3-benzo[b]thiophen-3-yl-azetidin-3-ol hydrochloride (68 mg, 4.06 mmol) and triethylamine (0.10 mL, 0.70 mmol) in dichloromethane (0.5 mL) at 3° C. The resulting mixture was allowed to warm to ambient temperature over 4 hours. The reaction mixture was diluted with dichloromethane (30 mL) and washed with dilute hydrochloric acid (20 mL, 0.1N) followed by saturated aqueous sodium chloride (20 mL). The organic layer was concentrated at reduce pressure. The resulting oil was dissolved in ethyl acetate (0.5 mL) and to this solution was added hydrogen chloride (1.5 mL, 4N in 1,4-dioxane) and the resulting solution was stirred for 5 hours. The reaction mixture was concentrated at reduced pressure and the resulting solid was purified by column chromatography through a silica gel cartridge (4 g) eluting with ethyl acetate/hexane (1:9 to 1:1) to give the product as a white solid (13 mg, 16% over 3 steps). ¹H NMR (DMSO-d₆) δ 8.01 (m, 1H,), 7.88 (m, 1H), 7.82 (s, 1H), 7.39 (m, 2H), 6.98 (s, 2H), 6.39 (s, 1H), 4.17 (d, J=8.3 Hz, 2H), 4.02 (d, J=8.3 Hz, 2H). MS(ES⁺)=285 (MH).

### Example 10

### Synthesis of (R)-2-(2-fluorophenyl)-2-hydroxyethylsulfamide

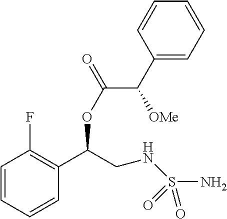

(S)-Methoxy-phenyl-acetic acid 2-sulfamide-(S)-1-(2-fluoro-phenyl)-ethyl ester. To a solution of N-[2-(2-fluoro-phenyl)-2-hydroxy-propyl]-sulfamide (1.5 g, 6.4 mmol), (S)-methoxy-phenyl-acetic acid (0.976 g, 8.4 mmol) in dichloromethane (20 mL) was added 1-ethyl-3-(3-dimethylaminopropyl) carbodiimide) (1.73 g, 9.04 mmol) and N,N-dimethylaminopyridine (79.3 mg, 0.65 mol). The reaction mixture was stirred for 18 hours, diluted with dichloromethane (30 mL) and washed with water. The organic layer was dried (anhydrous magnesium sulfate), concentrated and purified by column chromatography through a silica gel cartridge (120 g) eluting with ethyl acetate/toluene (0% to 15%) to give the two diasteromers (S)-methoxy-phenyl-acetic acid 2-sulfamide-(R)-1-(2-fluoro-phenyl)-ethyl ester, higher Rf compound (0.73 g, 30%, colorless oil); (S)-methoxy-phenyl-acetic acid 2-sulfamide-(S)-1-(2-fluoro-phenyl)-ethyl ester, lower Rf compound (1.23 g, 50%, colorless oil). ¹H NMR (CDCl₃) δ 7.40-7.31 (m, 5H), 7.21-7.12 (m, 1H), 6.96-6.78 (m, 3H), 6.17 (dd, J=11.0, 7.2 Hz, 1H), 5.10-4.98 (br s, 1H), 4.90-4.78 (br s, 2H), 4.88 (s, 1H), 3.38 (s, 3H), 3.21-3.10 (m, 2H), 2.20-2.02 (m, 2H).

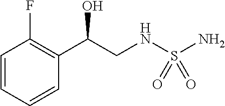

(S)-2-(2-Fluorophenyl)-2-hydroxyethylsulfamide. To a solution of (S)-methoxy-phenyl-acetic acid 2-sulfamide-(S)-1-(2-fluoro-phenyl)-ethyl ester (1.23 g, 3.23 mmol) in tetrahydrofuran (15 mL) at 0° C. was added lithium hydroxide (4.2 mL, 4.2 mmol, 1 N in water). The cooling bath was removed and the reaction mixture was stirred at room temperature for 1 hour. Ethyl acetate (30 mL) was added and the solution was washed with water (20 mL), dried (anhydrous magnesium sulfate), concentrated and purified through column chromatography through a silica gel cartridge (40 g) eluting with ethyl acetate/hexane (20% to 70%) to give the product as a colorless oil that crystallized on standing (610 mg, 81%). ¹H NMR (DMSO-d₆) δ 7.51-7.44 (m, 1H), 7.35-7.25 (m, 1H), 7.22-7.08 (m, 2H), 6.50 (s, 2H), 6.38 (t, J=6.5 Hz, 1H), 5.53 (d, J=4.7 Hz, 1H), 4.98-4.91 (m, 1H), 3.16-2.92 (m, 2H). MS(ES⁺)=217.05 (M−H₂O+H)⁺.

### Example 11

### Synthesis of (S)-2-(2-fluorophenyl)-2-hydroxyethylsulfamide

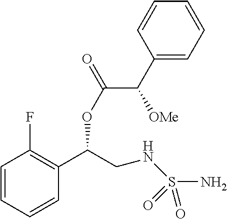

(S)-Methoxy-phenyl-acetic acid 2-sulfamide-(R)-1-(2-fluoro-phenyl)-ethyl ester was prepared by the same procedure as (S)-methoxy-phenyl-acetic acid 2-sulfamide-(S)-1-(2-fluoro-phenyl)-ethyl ester in example 287. ¹H NMR (CDCl₃) δ 0.26-7.33 (m, 4H), 7.31-7.16 (m, 2H), 7.05-6.88 (m, 3H), 6.22 (dd, J=7.6, 3.5 Hz, 1H), 4.90 (s, 1H), 4.70-4.50 (m, 3H), 3.69-3.55 (m, 1H), 3.52-2.95 (m, 4H).

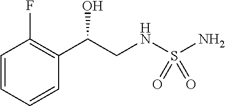

(R)-3-(2-Fluorophenyl)-2-hydroxyethylsulfamide was prepared by the same procedure as example 287. ¹H NMR (DMSO-d₆) δ 7.51-7.44 (m, 1H), 7.35-7.25 (m, 1H), 7.22-7.08 (m, 2H), 6.50 (s, 2H), 6.38 (t, J=6.3 Hz, 1H), 5.53 (d, J=4.7 Hz, 1H), 4.98-4.91 (m, 1H), 3.16-2.92 (m, 2H). MS(ES⁺)=217.05 (M−H₂O+H)⁺.

### Example 12

### Synthesis of (S)-2-(2-Chlorophenyl)-2-hydroxyethylsulfamide

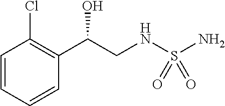

(S)-2-(2-Chlorophenyl)-2-hydroxyethylsulfamide was prepared by the same procedure as example 10. ¹H NMR (DMSO-d₆) δ 7.62-7.55 (m, 1H), 7.42-7.20 (m, 3H,), 6.45 (s, 2H), 6.35 (t, J=7.1 Hz, 1H), 5.60 (d, J=4.7 Hz, 1H), 5.08-4.96 (m, 1H), 3.20-3.05 (m, 1H), 2.90-2.80 (m, 1H). MS(ES⁺)=233.00 (M−H₂O+H)⁺.

### Example 13

### Synthesis of (R)-2-(2-Chlorophenyl)-2-hydroxyethylsulfamide

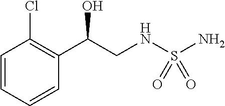

(R)-2-(2-Chlorophenyl)-2-hydroxyethylsulfamide was prepared by the same procedure as example 10. ¹H NMR (DMSO-d₆) δ 7.62-7.55 (m, 1H), 7.42-7.20 (m, 3H,), 6.45 (s, 2H), 6.35 (t, J=7.1 Hz, 1H), 5.60 (d, J=4.7 Hz, 1H), 5.08-4.96 (m, 1H), 3.20-3.05 (m, 1H), 2.90-2.80 (m, 1H). MS(ES⁺)=233.00 (M−H₂O+H)⁺.

### Procedures

The following procedures can be utilized in evaluating and selecting compounds as neuroprotective agents.

Cell cultures: All compounds are screened with dissociated hippocampal cultures derived from embryonic day 18 rats as the primary test system. With this preparation, primary neurons are used to test for toxicity as well as neuroprotection in a highly relevant experimental system to epilepsy. In brief, hippocampal tissue are obtained commercially through Brain Bits (Springfield, Ill.) and cultures prepared as previously described by Brewer (Brewer, G. J. Serum-free B27/neurobasal medium supports differentiated growth of neurons from the striatum, substantia nigra, septum, cerebral cortex, cerebellum and dentate gyms, J. Neurosci. Res. 1995, 42, 674-683.). The hippocampal neurons are platted at low density (10,000 cell/well) in a 96-well format and maintained in serum-free medium consisting of Neurobasal Medium supplemented with B27 and GlutaMAX (Gibco). Pre-coated poly-L-lysine coated plates are used because of the preferential adherence and survival of hippocampal neurons on this matrix support.

In Vitro Toxicity Testing:

Carboxyfluorescein (CFDA) was used a vital stain for all cell toxicity and neuroprotection studies. With the use of the CytoFluor fluorimeter, the CFDA assay was employed to assess the viability of neurons. CFDA is a dye that becomes fluorescent upon cell entry and cleavage by cytosolic esterases (Petroski, R. E.; Geller, H. M Selective labeling of embryonic neurons cultures on astrocyte monolayers with 5(6)-carboxyfluorescein diacetate (CFDA). J. Neurosci. Methods 1994, 52, 23-32.). Neuronal specificity is obtained relative to astrocytes because the cleaved dye is extruded extracellularly by glia with time, while dye in neurons remains intracellular. Previous experience with this assay showed a good correlation with neuronal cell counts stained immunocytochemically with neuron specific enolase antibodies, a reference marker for neuronal identity in complex cultures. To further assess the culture responses, a propidium iodide method was used as previously described (Sarafian, T. A.; Kouyoumjian, S.; Tashkin, D.; Roth, M. D. Synergistic cytotoxicity of 9-tetrahydrocannabianol and butylated hydroxyanisole, Tox. Letters, 2002. 133, 171-179.) to measure the number of dead cells. Propidium iodide becomes fluorescent when binding to the DNA of dead cells. Cultures were treated on day 2 with the test agent and then the two assays were conducted after a four day test period. For all assays, a 96-well format was used. For the screen, log concentration-effect studies were conducted from 10 nM to 1 mM with 8 replications. The duration of the test period was five days. Cultures were given a complete change of medium prior to the initiation of the treatment period.

Experimental details for the propidium iodide assay (Sarafian, T. A.; Kouyoumjian, S.; Tashkin, D.; Roth, M. D. Synergistic cytotoxicity of 9-tetrahydrocannabianol and butylated hydroxyanisole, Tox. Letters, 2002. 133, 171-179.): All test compounds were dissolved to 10 mM in Dulbecco's phosphate buffered saline (DPBS; Sigma:D-5780) prior to testing. On day two after plating, the test compound was added to the hippocampal cultures for a 4 day test period. Compounds were tested from 1 nM to 1 mM. At the conclusion of the test period, the cultures were tested for the amount of cell death by the propidium iodide method. Propidium iodide (PI) stock solution of 1 mg/ml (1.5 mM) was obtained from Sigma. The PI stock was diluted 1:30 in DPBS for a final working concentration of 50 μM. After removal of the growth medium, 50 μl of the 50 μM PI solution was added to cultures and allowed to incubate in the dark at room temperature for 15 min. The cultures were then assessed for fluorescence intensity at Ex536/Em590 nm in a CytoFluor fluorimeter. Results were expressed in relative fluorescent units and as a % of control values.

Experimental details for the CFDA assay (Petroski, R. E.; Geller, H. M Selective labeling of embryonic neurons cultures on astrocyte monolayers with 5(6)-carboxyfluorescein diacetate (CFDA). J. Neurosci. Methods 1994, 52, 23-32.): All test compounds were dissolved to 10 mM in Dulbecco's phosphate buffered saline (DPBS; Sigma:D-5780) prior to testing. On day two after plating, the test compound was added to the hippocampal cultures for a 4 day test period. Compounds were tested from 1 nM to 1 mM. At the conclusion of the test period, the cultures were tested for the amount of neuronal viability by the CFDA method. For the neuronal viability assay, 1 mg of 5,6-carboxyfluorescein diacetate (CFDA) dye (Sigma) was dissolved in 100 ml of DPBS (Gibco:D-5780) and kept in the dark until added to the hippocampal cultures. After a complete change of medium of day 5 hippocampal test cultures, 100 μl CFDA dye solution was added for 15 min of incubation at 37° C. in the dark. At the conclusion of the incubation period, the dye was removed from the cultures and washed once with 100 μl of DPBS. After removal of the first wash, a second wash of DPBS was added to the culture and then incubated for 30 min to allow the efflux of dye out of glia in the cultures. At the conclusion of the 30 min efflux period, the culture efflux medium was removed and 100 μl of 0.1% triton-X100 in water was added to the cultures before reading at Ex490/Em517 in a CytoFluor fluorimeter. Results were expressed in relative fluorescent units (RFU).

Neuroprotection Assays:

Potent neuroprotection is the distinguishing characteristic that separates this program's anticonvulsants from all other commercial drugs for epilepsy. The experimental details and the rationale for the implemented assays are essential in differentiating these compounds from that of others. The central objective of all neuroprotective assays was their relevancy to excitotoxicity and oxidative stress related to epilepsy. Both the amount of glutamate and hydrogen peroxide used in the assays, as well as the time of treatment and duration of the experiment, were designed to be relevant to epilepsy. Further, all time parameters employed in these studies were empirically determined to be within the limits of reversible toxic events, yet using amounts of glutamate and hydrogen peroxide that were relevant to the disease. In regard to glutamate toxicity, a critical feature was the duration of treatment of the hippocampal neurons. The rational for using a short 5 min treatment with glutamate was based on the observation of Randall and Thayer (Randall, R. D.; Thayer, S. A. Glutamate-induced calcium transient triggers calcium overload and neurotoxicity in rat hippocampal neurons, J. Neurosci. 1992, 12, 1882-1895). Their study demonstrated that a short-term treatment with glutamate produced a delayed but substantial increase in intracellular calcium that overloaded the neuron and produced cell death. The rationale is that this intense burst of glutamate and resulting calcium overload is relevant to seizures and therefore was important data to capture in the screening assay. The amount of glutamate (30 μM) employed in our screening was based on the basal levels of glutamate observed in microdialysis measurements of hippocampus from epileptogenic patients (Cavus et al. Decreased hippocampal volume on MRI is associated with increased extracellular glutamate in epilepsy patients, Epilepsia, 2008, 49, 1358-1366.). In regard to hydrogen peroxide, the amount employed (10 μM) was detected in the hippocampus of rats after kainate-induced status epilepticus (Jarrett et. al., Mitochondrial DNA damage and impaired base excision repair during epileptogenesis, Neurobiol. Dis. 2008, 30, 130-138). To produce neural damage and death with these amounts of glutamate and hydrogen peroxide, the cultures were changed to a medium with significant depletion of antioxidant components in the defined medium supplement B-27 just prior to treatment with the compounds. This was performed to obtain a significant and reproducible toxic signal in the hippocampal neurons and because loss of antioxidant control may be a component of epileptogenesis (Waldbaum and Patel, Mitochondria, oxidative stress and temporal lobe epilepsy, Epilepsy Res. 2010 88, 23-45.; Wu et al., Mitochondrial DNA mutation-elicited oxidative stress, oxidative damage, and altered gene expression in cultured cells of patients with MERRF syndrome, Mol. Neurobiol. 2010, 41, 256-266.). Neuroprotection studies with hydrogen peroxide were conducted with cultures that were between day 12 and day 18. Studies of neuroprotection glutamate were conducted between day 19 and day 22. Assays for neuronal viability and cell death were identical to those described in the cell toxicity section.

Experimental Details of the Propidium Iodide Neuroprotection Assay:

Neuroprotection from oxidative stress: All test compounds are dissolved to 10 mM in Dulbecco's phosphate buffered saline (DPBS; Sigma:D-5780) prior to testing. To test for neuroprotection from hydrogen peroxide, day 11 hippocampal cultures are given a complete change of medium containing 100 μl of Neurobasal medium with B27 that contains no antioxidants. Twenty-four hours after the change in medium, the hydrogen peroxide neuroprotection studies are started. The test compound is added to the hippocampal cultures for a 4 hour test period in concentrations that ranged from 1 nM to 300 μM. Concurrent with the treatment of test compound, 10 μM hydrogen peroxide is added for the 4 hour test period. At the conclusion of the test period, the cultures are tested for the amount of cell death by the propidium iodide method. Propidium iodide (PI) stock solution of 1 mg/ml (1.5 mM) is obtained from Sigma. The PI stock is diluted 1:30 in DPBS for a final working concentration of 50 μM. After removal of the growth medium, 50 μl of the 50 μM PI solution is added to cultures and allowed to incubate in the dark at room temperature for 15 min. The cultures are then assessed for fluorescence intensity at Ex536/Em590 nm in a CytoFluor fluorimeter. Results are expressed in relative fluorescent units and EC₅₀'s calculated from the dose response of the test compound.

Neuroprotection from Excitotoxicity:

For the glutamate neuroprotection studies with the propidium iodide assay, several modifications are made from the method described for the hydrogen peroxide assay. For the glutamate neuroprotection assay, day 19 hippocampal cultures are given a complete change of medium containing 100 μl of Neurobasal medium with B27 that contained no antioxidants. Twenty-four hours after the change in medium, the glutamate neuroprotection studies are started. The day 20 cultures are treated for 5 min with 30 μM glutamate dissolved in DPBS. For this treatment, a 900 μM solution of glutamate is prepared and then 3.3 μL of this solution is added to the culture well containing 100 μL of media. After this short treatment, the medium containing the glutamate is removed from the cultures and fresh medium with antioxidants added. The test compound is then added to the hippocampal cultures for a 4 hour test period in concentrations that ranged from 1 nM to 300 μM. At the conclusion of the test period, the cultures are tested for the amount of cell death by the propidium iodide method. Propidium iodide (PI) stock solution of 1 mg/ml (1.5 mM) is obtained from Sigma. The PI stock is diluted 1:30 in DPBS for a final working concentration of 50 μM. After removal of the growth medium, 50 μl of the 50 μM PI solution is added to cultures and allowed to incubate in the dark at room temperature for 15 min. The cultures are then assessed for fluorescence intensity at Ex536/Em590 nm in a CytoFluor fluorimeter. Results are expressed in relative fluorescent units and EC₅₀'s calculated from the dose response of the test compound.

Experimental Details of the CFDA Neuroprotection Assay:

Neuroprotection from Oxidative Stress:

All test compounds are dissolved to 10 mM in Dulbecco's phosphate buffered saline (DPBS; Sigma:D-5780) prior to testing. To test for neuroprotection from hydrogen peroxide, day 11 hippocampal cultures are given a complete change of medium containing 100 μl of Neurobasal medium with B27 that contained no antioxidants. Twenty-four hours after the change in medium, the hydrogen peroxide neuroprotection studies are started. The test compound is added to the day 12 hippocampal cultures for a 4 hour test period in concentrations that ranged from 1 nM to 300 μM. Concurrent with the treatment of test compound, 10 μM hydrogen peroxide is added for the 4 hour test period. At the conclusion of the test period, the cultures are tested for the amount of neuronal viability by the CFDA method. For the neuronal viability assay, 1 mg of 5,6-carboxyfluorescein diacetate (CFDA) dye (Sigma) is dissolved in 100 ml of DPBS (Gibco:D-5780) and kept in the dark until added to the hippocampal cultures. After a complete change of medium of day 12 hippocampal test cultures, 100 μl CFDA dye solution is added for 15 min of incubation at 37° C. in the dark. At the conclusion of the incubation period, the dye is removed from the cultures and washed once with 100 μl of DPBS. After removal of the first wash, a second wash of DPBS is added to the culture and then incubated for 30 min to allow the efflux of dye out of glia in the cultures. At the conclusion of the 30 min efflux period, the culture efflux medium is removed and 100 μl of 0.1% triton-X100 in water is added to the cultures before reading at Ex490/Em517 in a CytoFluor fluorimeter. Results are expressed in relative fluorescent units (RFU) and EC₅₀'s calculated from the dose response of the test compound.

Neuroprotection from Excitotoxicity:

For the glutamate neuroprotection studies with the CFDA assay, several modifications are made from the method described for the hydrogen peroxide assay. For the glutamate neuroprotection assay, day 19 hippocampal cultures are given a complete change of medium containing 100 μl of Neurobasal medium with B27 that contained no antioxidants. Twenty-four hours after the change in medium, the glutamate neuroprotection studies are started. The day 20 cultures are treated for 5 min with 30 μM glutamate dissolved in DPBS. For this treatment, a 900 μM solution of glutamate is prepared and then 3.3 μL of this solution is added to the culture well containing 100 L of media. After this short treatment, the medium containing the glutamate is removed from the cultures and fresh medium with antioxidants added. The test compound is then added to the hippocampal cultures for a 4 hour test period in concentrations that ranged from 1 nM to 300 μM At the conclusion of the test period, the cultures are tested for the amount of neuronal viability by the CFDA method. For the neuronal viability assay, 1 mg of 5,6-carboxyfluorescein diacetate (CFDA) dye (Sigma) is dissolved in 100 ml of DPBS (Gibco:D-5780) and kept in the dark until added to the hippocampal cultures. After a complete change of medium of day 20 hippocampal test cultures, 100 μl CFDA dye solution is added for 15 min of incubation at 37° C. in the dark. At the conclusion of the incubation period, the dye is removed from the cultures and washed once with 100 μl of DPBS. After removal of the first wash, a second wash of DPBS is added to the culture and then incubated for 30 min to allow the efflux of dye out of glia in the cultures. At the conclusion of the 30 min efflux period, the culture efflux medium is removed and 100 μl of 0.1% triton-X100 in water is added to the cultures before reading at Ex490/Em517 in a CytoFluor fluorimeter. Results are expressed in relative fluorescent units (RFU) and EC₅₀'s calculated from the dose response of the test compound. Results are expressed in relative fluorescent units and EC₅₀'s calculated from the dose response of the test compound.

Methods to measure anticonvulsant activity: All tests were conducted by the Anticonvulsant Screening Program (ASP) of the National Institute of Neurological Disease and Stroke at the National Institutes of Health.

The Maximal Electroshock Seizure (MES) or Maximal Seizure Pattern Test: The MES is a model for generalized tonic-clonic seizures (Putnam and Merritt, 1937). It is highly reproducible with consistent endpoints. The behavioral and electrographic seizures generated in this model are consistent with the human disorder. This model identifies those compounds which prevent seizure spread.

In the MES test, an electrical stimulus of 0.2 s in duration (50 mA in mice and 150 mA in rat at 60 Hz) is delivered via corneal electrodes primed with an electrolyte solution containing an anesthetic agent. Mice are tested at 30 minutes and 4 hours following doses of 30, 100 and 300 mg/kg of test compound. Test compounds are administered intraperitoneally to the mice. Rats are tested at time intervals between 0.25 and 4 hours following a standard oral dose of 30 mg/kg. Abolition of the hindlimb tonic extensor component indicates the test compound's ability to inhibit MES-induced seizure spread.

The 6 Hz Seizure Test: Compounds were further tested in the 6-Hz psychomotor seizure model (Barton et al., 2001). This model is used to detect seizures that may be useful for the treatment of therapy-resistant partial seizures. For this test, an alternative electrical stimulation paradigm is used with a low frequency (6 Hz), long duration (3 seconds) corneal stimulation. The seizure evoked by this low frequency stimulation is characterized by immobility, forelimb clonus, Straub tail and facial automatisms (Barton et al., 2001). Mice are tested from 15 min to 4 hours following an intraperitoneal dose of 100 mg/kg. Abolition of the clonic seizures indicates the ability of a compound to prevent partial or psychomotor seizures.

Results for representative compounds according to the present invention are listed in Table 1 and 2 below.

TABLE 1
Examples of sulfamide compounds and their in vitro potencies

| | | NP* from | NP* from | | |
|---|---|---|---|---|---|
| | | Glutamate | Glutamate | NP** from | NP** from |
| Example | | PI | CFDA | HP PI | HP CFDA |
| number | Structures | EC₅₀ | | | |
| 1 | 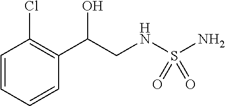 | 100 μM | 30 μM | | |
| 2 | 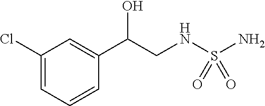 | >300 μM | >300 μM | 100 μM | 100 μM |
| 3 | 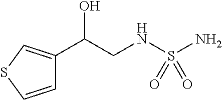 | 30 μM | >300 μM | 30 μM | 30 μM |
| 4 | 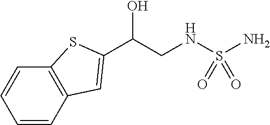 | 10 μM | 100 μM | 30 μM | 100 μM |
| 5 | 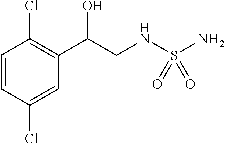 | 30 μM | >300 μM | >300 μM | >300 μM |
| 6 | 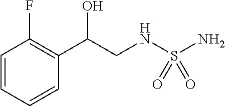 | 10 μM | 100 μM | 10 nM | 10 μM |
| 7 | 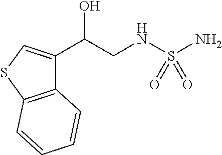 | >300 μM | 30 μM | | |
| 8 | 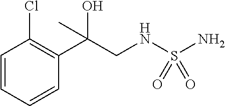 | 300 μM | >300 μM | 100 nM | 100 μM |
| 9 | 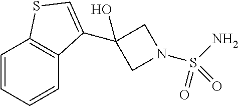 | 1 μM | 100 nM | >300 μM | >300 μM |
| 10 | 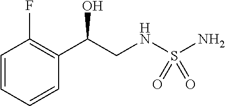 | 100 nM | 300 μM | 100 nM | 1 μM |
| 11 | 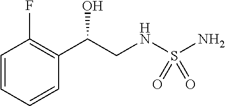 | | | 300 μM | 300 μM |
| 12 | 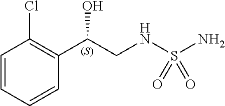 | 10 μM | 30 μM | >300 μM | 30 μM |
| 13 | 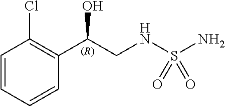 | 10 μM | 10 nM | >300 μM | 300 μM |

*NP = Neuroprotection from 30 μM glutamate in Hippocampal Cultures
**NP = Neuroprotection from 10 μM Hydrogen Peroxide in Hippocampal Cultures

TABLE 2
Selective examples of sulfamide compounds and their in vivo potencies*

| | | Dose | | # animals active/ |
|---|---|---|---|---|
| Assay | Compound # | (mg/kg) | Time | # animals tested |
| Mouse MES** Mouse MES** Mouse MES** Rat MES** Rat MES** Mouse 6 Hz Mouse 6 Hz Mouse 6 Hz Mouse 6 Hz | 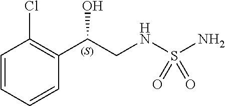 | 30 100 300 30 30 100 100 100 100 | 0.5 hr 0.5 hr 0.5 hr 0.25 hr 0.5 hr 0.25 hr 0.5 hr 1.0 hr 2.0 hr | 0/1 2/3 1/1 1/4 0/4 0/4 1/4 1/4 0/4 |
| Mouse MES** Mouse MES** Mouse MES** Rat MES** Rat MES** Rat MES** Mouse 6 Hz Mouse 6 Hz Mouse 6 Hz Mouse 6 Hz Mouse 6 Hz | 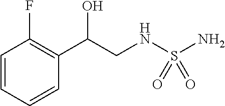 | 30 100 300 30 30 30 100 100 100 100 100 | 4 hr 4 hr 4 hr 1 hr 2 hr 4 hr 0.25 hr 0.5 hr 1.0 hr 2.0 hr 4.0 hr | 0/1 1/3 1/1 0/4 1/4 2/4 0/4 1/4 1/4 1/4 1/4 |

*Vehicle: methylcellulose
**MES = Maximal Electroshock Test in mice (anti-seizure test)

1. A compound represented by formula (I): 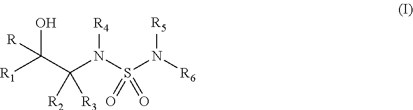
and hydrates, solvates, pharmaceutically acceptable salts, prodrugs and complexes thereof, wherein:
R is a member selected from the group consisting of optionally substituted aryl, heterocyclyl, heteroaryl, and aliphatic, where R may be substituted by 0-5 moieties including, but not limited to aliphatic, hydroxyl, alkoxy amino, nitro, cyano, and halogen;
R₁, R₂, R₃, R₄, R₅ and R₆ are each members independently selected from the group consisting of hydrogen and aliphatic;
R₁ and R₂ or R₃ or R₄ optionally form a ring of 4-8 atoms, which includes at least one of C, N, O, and S;
R₂ and R₃ optionally form a ring of 4-8 atoms, which includes at least one of C, N, O, and S;
R₂ or R₃ and R₄ optionally form a ring of 4-8 atoms, which includes at least one of C, N, O, and S; and
R₄ and R₅ or R₆ optionally form a ring of 4-8 atoms, which includes at least one of C, N, O, and S.

2. The compound according to claim 1 wherein R is 2-chlorophenyl, 3-chlorophenyl, thiophen-3-yl, benzothiophen-2-yl, 2,5-dichlorophenyl, 2-fluorophenyl, or benzothiophen-3-yl.

3. The compound according to claim 1 wherein R₁ is methyl or hydrogen.

4. The compound according to claim 1 wherein R₁ and R₄ are part of a ring of 4 atoms.

5. The compound according to claim 1 that is:
2-(2-Chlorophenyl)-2-hydroxyethylsulfamide;
2-(3-Chlorophenyl)-2-hydroxyethylsulfamide;
2-(thiophen-3-yl)-2-hydroxyethylsulfamide;
2-(benzothiophen-2-yl)-2-hydroxyethylsulfamide;
2-(2,5-dichlorophenyl)-2-hydroxyethylsulfamide;
2-(2-fluorophenyl)-2-hydroxyethylsulfamide;
2-(benzothiophen-3-yl)-2-hydroxyethylsulfamide;
2-(2-chlorophenyl)-2-methyl-2-hydroxyethylsulfamide;
3-(benzo[b]thiophen-3-yl)-3-hydroxy-azetidine-1-sulfamide;
(R)-2-(2-fluorophenyl)-2-hydroxyethylsulfamide
(S)-2-(2-fluorophenyl)-2-hydroxyethylsulfamide;
(S)-2-(2-Chlorophenyl)-2-hydroxyethylsulfamide;
(R)-2-(2-Chlorophenyl)-2-hydroxYethylsulfamide;
or a pharmaceutically acceptable form thereof.

6. A composition comprising an effective amount of at least one compound according to claim 1.

7. A composition according to claim 6, further comprising at least one excipient.

8. A composition according to claim 7, wherein the at least one compound is at least one member selected from the group consisting of
2-(2-Chlorophenyl)-2-hydroxyethylsulfamide;
2-(3-Chlorophenyl)-2-hydroxyethylsulfamide;
2-(thiophen-3-yl)-2-hydroxyethylsulfamide;
2-(benzothiophen-2-yl)-2-hydroxyethylsulfamide;
2-(2,5-dichlorophenyl)-2-hydroxyethylsulfamide;
2-(2-fluorophenyl)-2-hydroxyethylsulfamide;
2-(benzothiophen-3-yl)-2-hydroxyethylsulfamide;
2-(2-chlorophenyl)-2-methyl-2-hydroxyethylsulfamide;
3-(benzo[b]thiophen-3-yl)-3-hydroxy-azetidine-1-sulfamide;
(R)-2-(2-fluorophenyl)-2-hydroxyethylsulfamide
(S)-2-(2-fluorophenyl)-2-hydroxyethylsulfamide;
(S)-2-(2-Chlorophenyl)-2-hydroxyethylsulfamide;
(R)-2-(2-Chlorophenyl)-2-hydroxyethylsulfamide; and
pharmaceutically acceptable forms thereof.

9. A method for treating a disease associated with excessive glutamate, said method comprising administering to a subject an effective amount of at least one compound according to claim 1.

10. The method of claim 9, wherein the at least one compound is administered in a composition further comprising at least one excipient.

11. The method of claim 10, wherein the at least one compound is at least one member selected from the group consisting of
2-(2-Chlorophenyl)-2-hydroxyethylsulfamide;
2-(3-Chlorophenyl)-2-hydroxyethylsulfamide;
2-(thiophen-3-yl)-2-hydroxyethylsulfamide;
2-(benzothiophen-2-yl)-2-hydroxyethylsulfamide;
2-(2,5-dichlorophenyl)-2-hydroxyethylsulfamide;
2-(2-fluorophenyl)-2-hydroxyethylsulfamide;
2-(benzothiophen-3-yl)-2-hydroxyethylsulfamide;
2-(2-chlorophenyl)-2-methyl-2-hydroxyethylsulfamide;
3-(benzo[b]thiophen-3-yl)-3-hydroxy-azetidine-1-sulfamide;
(R)-2-(2-fluorophenyl)-2-hydroxyethylsulfamide
(S)-2-(2-fluorophenyl)-2-hydroxyethylsulfamide;
(S)-2-(2-Chlorophenyl)-2-hydroxyethylsulfamide;
(R)-2-(2-Chlorophenyl)-2-hydroxyethylsulfamide; and
pharmaceutically acceptable forms thereof.

12. The method of claim 11, wherein the disease is a neurodegenerative disease or epilepsy.

13. The method of claim 11, wherein the disease is Alzheimer's disease.

14. The method of claim 10, wherein the disease is a neurodegenerative disease or epilepsy.

15. The method of claim 10, wherein the disease is Alzheimer's disease.
